(19) United States
(12) Patent Application Publication
(10) Pub. No.: US 2021/0151193 A1
(43) Pub. Date: May 20, 2021

(54) Executing Temporally Dynamic Medical Event Processing

(71) Applicant: Optum, Inc., Minnetonka, MN (US)

(72) Inventors: Jon Kevin Muse, Thompson's Station, TN (US); Gregory J. Boss, Saginaw, MI (US); Rama S. Ravindranathan, Edison, NJ (US)

(21) Appl. No.: 16/685,109

(22) Filed: Nov. 15, 2019

(51) Int. Cl. G16H 50/30; G16H 10/60; G06F 9/54; G06Q 10/10

(52) U.S. Cl. CPC G16H 50/30; G06Q 10/1097; G06F 9/542; G16H 10/60

(57) ABSTRACT

There is a need for more effective and efficient medical event processing. This need can be addressed by, for example, solutions for performing/executing performing temporally dynamic medical event processing. In one example, a method comprises determining a medical need condition of a patient profile; determining a temporally dynamic service quality model for each available service option associated with the patient profile; determining, based at least in part on the medical need condition and each temporally dynamic service quality model for an available service option of, an optimal service option for the medical need condition; and performing service delivery actions by communicating with at least one of a patient computing entity associated with the patient profile and a provider computing entity associated with the optimal service option.

### BACKGROUND

Various embodiments of the present invention address technical challenges related to performing medical event processing. Medical event processing is an example of a real-time event processing task that presents unique and complex efficiency and reliability challenges because of its predictive complexity and sensitivity. Various embodiments of the present address the shortcomings of the noted medical event processing systems and disclose various techniques for efficiently and reliably performing medical event processing.

### BRIEF SUMMARY

In general, embodiments of the present invention provide methods, apparatus, systems, computing devices, computing entities, and/or the like for performing/executing temporally dynamic medical event processing. Certain embodiments utilize systems, methods, and computer program products that perform/execute temporally dynamic medical event processing using one or more of contextually aware medical need detections, temporally dynamic service quality models, static characterizations of current time intervals associated with medical need scenarios, and dynamic characterizations of current time intervals associated with medical need scenarios.

In accordance with one aspect, a method is provided. In one embodiment, the method comprises determining, by one or more processors, a medical need condition of a patient profile associated with the medical need scenario, wherein (i) the medical need condition is selected from a plurality of candidate medical need conditions, (ii) each of the plurality of candidate medical need conditions is characterized by one or more medical response expectations comprising a patient competence expectation, and (iii) determining the medical need condition is based at least in part on a patient competence state associated with the medical need scenario; determining, by the one or more processors, a temporally dynamic service quality model for each available service option of a plurality of available service options associated with the patient profile, wherein: (i) the corresponding temporally dynamic service quality model for an available service option indicates a current estimated service quality score for the available service option with respect to each candidate medical need condition of the plurality of candidate medical need conditions, and (ii) determining the temporally dynamic service quality model for an available service option comprises assigning a minimal current estimated service quality score for the available service option with respect to each candidate medical need condition of a plurality of candidate medical need conditions in response to determining that a transport capability designation of the available service option does not satisfy the patient competence expectation of the candidate medical need condition; determining, by the one or more processors and based at least in part on the medical need condition and each temporally dynamic service quality model for an available service option of the plurality of available service options, an optimal service option of the plurality of available service options; and performing, by the one or more processors, one or more service delivery actions by communicating with at least one of a patient computing entity associated with the patient profile or a provider computing entity associated with the optimal service option.

In accordance with another aspect, a computer program product is provided. The computer program product may comprise at least one computer-readable storage medium having computer-readable program code portions stored therein, the computer-readable program code portions comprising executable portions configured to determine, by one or more processors, a medical need condition of a patient profile associated with the medical need scenario, wherein (i) the medical need condition is selected from a plurality of candidate medical need conditions, (ii) each of the plurality of candidate medical need conditions is characterized by one or more medical response expectations comprising a patient competence expectation, and (iii) determining the medical need condition is based at least in part on a patient competence state associated with the medical need scenario; determine, by the one or more processors, a temporally dynamic service quality model for each available service option of a plurality of available service options associated with the patient profile, wherein: (i) the corresponding temporally dynamic service quality model for an available service option indicates a current estimated service quality score for the available service option with respect to each candidate medical need condition of the plurality of candidate medical need conditions, and (ii) determining the temporally dynamic service quality model for an available service option comprises assigning a minimal current estimated service quality score for the available service option with respect to each candidate medical need condition of a plurality of candidate medical need conditions in response to determining that a transport capability designation of the available service option does not satisfy the patient competence expectation of the candidate medical need condition; determine, by the one or more processors and based at least in part on the medical need condition and each temporally dynamic service quality model for an available service option of the plurality of available service options, an optimal service option of the plurality of available service options; and perform, by the one or more processors, one or more service delivery actions by communicating with at least one of a patient computing entity associated with the patient profile or a provider computing entity associated with the optimal service option.

In accordance with yet another aspect, an apparatus comprising at least one processor and at least one memory including computer program code is provided. In one embodiment, the at least one memory and the computer program code may be configured to, with the processor, cause the apparatus to determine, by one or more processors, a medical need condition of a patient profile associated with the medical need scenario, wherein (i) the medical need condition is selected from a plurality of candidate medical need conditions, (ii) each of the plurality of candidate medical need conditions is characterized by one or more medical response expectations comprising a patient competence expectation, and (iii) determining the medical need condition is based at least in part on a patient competence state associated with the medical need scenario; determine, by the one or more processors, a temporally dynamic service quality model for each available service option of a plurality of available service options associated with the patient profile, wherein: (i) the corresponding temporally dynamic service quality model for an available service option indicates a current estimated service quality score for the available service option with respect to each candidate medical need condition of the plurality of candidate medical need conditions, and (ii) determining the temporally dynamic service quality model for an available service option comprises assigning a minimal current estimated service quality score for the available service option with respect to each candidate medical need condition of a plurality of candidate medical need conditions in response to determining that a transport capability designation of the available service option does not satisfy the patient competence expectation of the candidate medical need condition; determine, by the one or more processors and based at least in part on the medical need condition and each temporally dynamic service quality model for an available service option of the plurality of available service options, an optimal service option of the plurality of available service options; and perform, by the one or more processors, one or more service delivery actions by communicating with at least one of a patient computing entity associated with the patient profile or a provider computing entity associated with the optimal service option.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example medical event processing computing entity in accordance with some embodiments discussed herein.

FIG. 3 provides an example patient computing entity in accordance with some embodiments discussed herein.

FIG. 4 provides an example provider computing entity in accordance with some embodiments discussed herein.

FIG. 5 is a flowchart diagram of an example process for performing/executing temporally dynamic medical event processing in accordance with some embodiments discussed herein.

FIG. 6 is a flowchart diagram of an example process for generating a temporally dynamic service quality model for an available service option in accordance with some embodiments discussed herein.

FIG. 7 is a flowchart diagram of an example process for determining a medical need condition in accordance with some embodiments discussed herein.

FIG. 8 is an operational example of a provider operational readiness user interface in accordance with some embodiments discussed herein.

FIG. 9 is an operational example of a medical need service availability user interface in accordance with some embodiments discussed herein.

FIG. 10 is an operational example of a static quality score distribution user interface in accordance with some embodiments discussed herein.

FIG. 11 is an operational example of a dynamic quality score distribution user interface in accordance with some embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. OVERVIEW

Medical event processing is an example of a real-processing event task which presents some unique efficiency and reliability challenges not addressed by current real-time processing models and literature. For example, medical event processing typically requires performing multiple sets of predictive inferences configured to detect medical conditions as well as multiple sets of predictive inferences configured to identify medical service options. Each of the noted sets of predictive inferences is a predictively complex task that may require processing large quantities of available input data with a complex input structure. Further contributing to this predictive complexity are problems associated with limited availability of optimal input data, time constraints typically imposed on medical event processing because of the time sensitivity of the underlying real-world conditions, and enhanced accuracy requirements because of a lower margin of error and higher penalties for wrong determinations. Because of the noted challenges, many existing medical event processing systems suffer from substantial efficiency and reliability challenges. Thus, there is a continuing technical need for improving efficiency and reliability of medical event processing systems.

To address the noted technical challenges associated with efficiency and reliability of medical event processing systems, various embodiments of the present invention disclose performing/executing temporally dynamic medical event processing. In one aspect of performing/executing temporally dynamic medical event processing relates to reducing the need for real-time processing of patient data and provider data in determining patient needs and operational readiness of available providers to address the detected patient needs. Of note, various embodiments of the performing/executing temporally dynamic medical event processing concepts accomplish a reduction in real-time processing need without ignoring temporal properties of medical need scenarios. In doing so, the noted embodiments reduce computational complexity of medical event processing routines without comprising accuracy of such routines to the extent such accuracy is a result of integrating temporal properties of medical need scenarios into predictive inferences.

For example, according to some embodiments, a temporally dynamic medical event processing system generates temporally dynamic service quality models for service providers based in part on static data determined entirely based at least in part on relating historic operational trends of the service providers to static properties of a time interval associated with a medical need scenario (e.g., based at least in part on time-of-day, date-of-week, and/or the like). Such static generation of service quality models decreases the need for real-time processing to generate service quality models without ignoring temporally unique properties of medical need scenarios. As another example, according to some embodiments, a temporally dynamic medical event processing system generates temporally dynamic service quality models for service providers based in part on static data determined entirely based at least in part on relating historic operational trends of the service providers to dynamic properties of a time interval associated with a medical need scenario, where the dynamic properties are determined based at least in part on current operational trends of the service providers. Such utilization of historic operational data in processing current operational trends provides another mechanism for decreasing the need for real-time processing to generate service quality models without ignoring temporally unique properties of medical need scenarios.

Using the above-described techniques and related techniques described throughout this document, various embodiments of the temporally dynamic medical event processing concepts accomplish a reduction in real-time processing need without ignoring temporal properties of medical need scenarios. In doing so, the noted embodiments reduce computational complexity of medical event processing routines without comprising accuracy of such routines to the extent such accuracy is a result of integrating temporal properties of medical need scenarios into predictive inferences. Accordingly, by utilizing the various temporally dynamic medical event processing techniques described herein, various embodiments of the present invention improve efficiency and reliability of medical event processing systems and make important technical contributions to medical event processing field and to the broader field of real-time event processing.

### II. COMPUTER PROGRAM PRODUCTS, METHODS, AND COMPUTING ENTITIES

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### III. Exemplary System Architecture

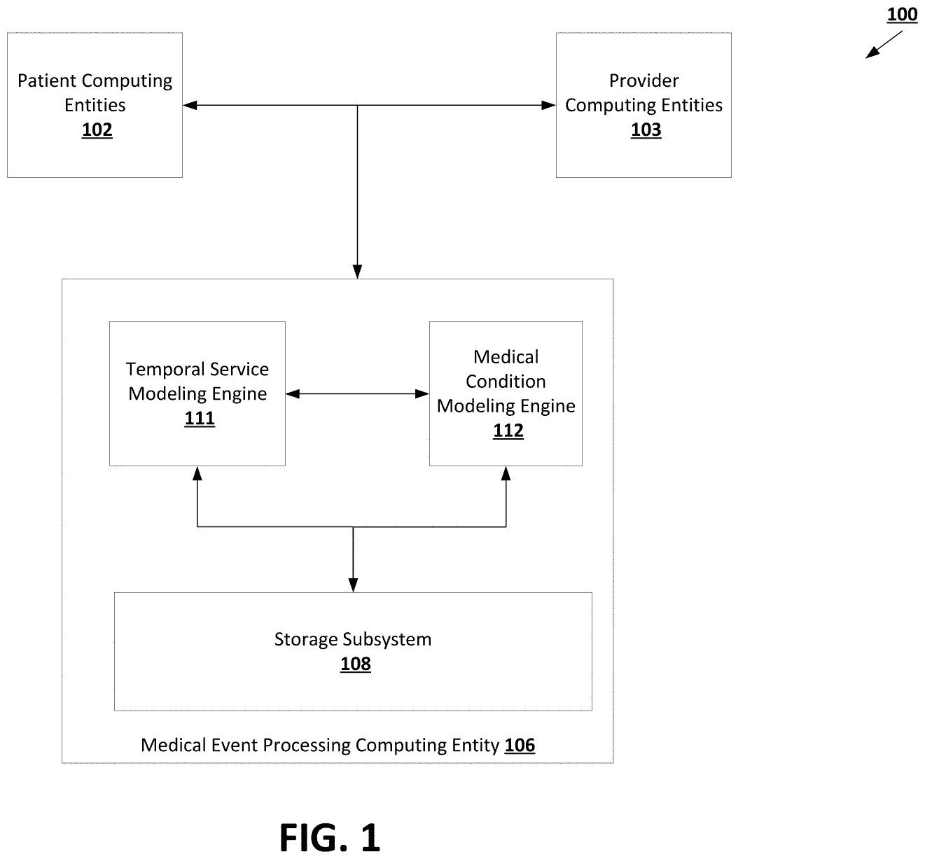

FIG. 1 is a schematic diagram of an example architecture 100 for performing/executing temporally dynamic medical event processing. The architecture 100 includes a medical event processing computing entity 106 configured to process patient condition data received from one or more patient computing entities 102 and provider condition data received from one or more provider computing entities 103 to assign optimal service options (e.g., medical providers) to particular patients given patient medical conditions and service quality models of candidate service options. The medical event processing computing entity 106 may further be configured to perform one or more prediction-based actions (e.g., automated appointment scheduling and/or ambulance scheduling) based at least in part on patient-service option assignments.

In some embodiments, the medical event processing computing entity 106 may communicate with at least one of the patient computing entities 102 and the provider computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

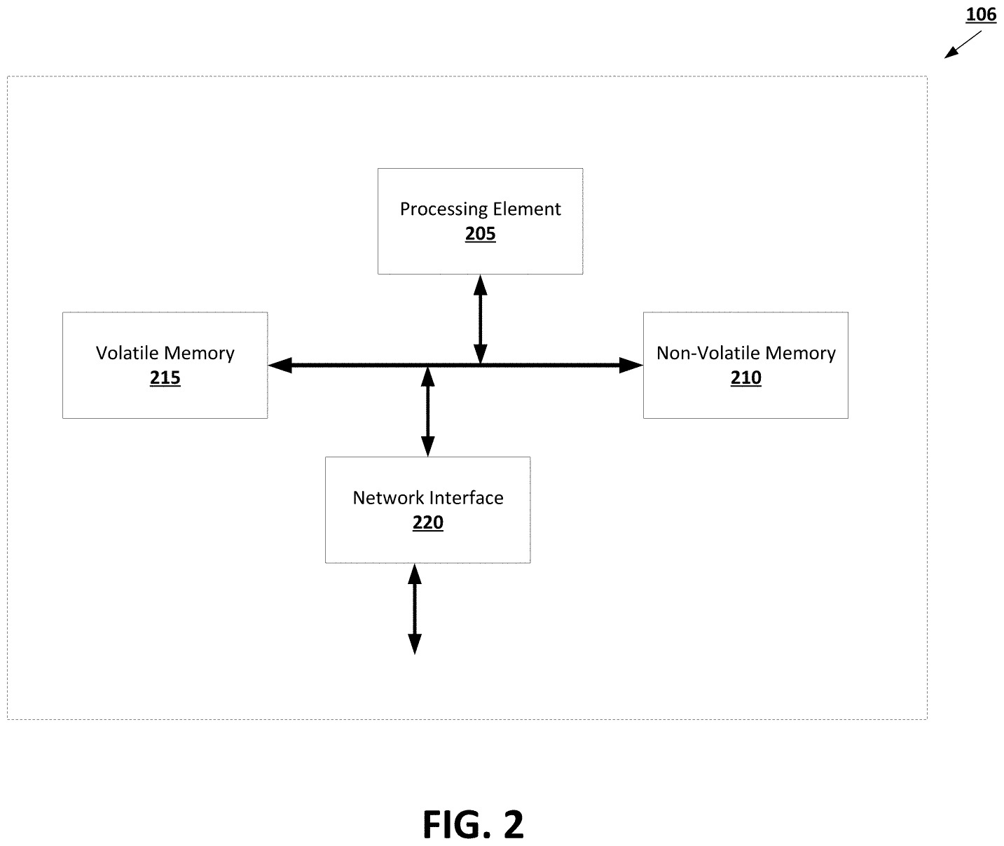

A patient computing entity 102 may be any computing entity that provides data about patient conditions and/or patient environment conditions. Examples of patient computing entities 102 include smartphones, smart watches, tablets, personal computers, sensory computing entities installed on patient bodies, sensory computing entities installed in patient environments, and/or the like. A provider computing entity 103 may be any computing entity that provides current or historic data about operation of a medical service option and/or environment of a medical service option facility. Examples of provider computing entities 103 include medical provider servers, municipal database servers, traffic management servers, and/or the like. Exemplary architectures for the medical event processing computing entity 106, the patient computing entities 102, and the provider computing entities are described below with reference to FIGS. 2-4.

The medical event processing computing entity 106 includes a temporal service modeling engine 111, a medical condition modeling engine 112, and a storage subsystem 108. The temporal service modeling engine 111 is configured to generate an optimal service option for a patient profile given a detected patient conduction and perform one or more prediction-based actions based at least in part on the prediction of the optimal service option. The medical condition modeling engine 112 is configured to detect a medical need scenario and a medical need condition associated with the medical need condition. The storage subsystem 108 is configured to store model definition data for at least one of the temporal service modeling engine 111 and a medical condition modeling engine 112 as well as historic operational data associated with past operations of candidate service options whose relevant data is maintained by the medical event processing computing entity 106. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Medical Event Processing Computing Entity

FIG. 2 provides a schematic of a medical event processing computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the medical event processing computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the medical event processing computing entity 106 may include or be in communication with one or more processing elements 205 (also referred to as processors, processing circuitry, and/or similar terms used herein interchangeably) that communicate with other elements within the medical event processing computing entity 106 via a bus, for example. As will be understood, the processing element 205 may be embodied in a number of different ways. For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the medical event processing computing entity 106 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the medical event processing computing entity 106 may further include or be in communication with volatile media (also referred to as volatile storage, memory, memory storage, memory circuitry and/or similar terms used herein interchangeably). In one embodiment, the volatile storage or memory may also include one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the medical event processing computing entity 106 with the assistance of the processing element 205 and operating system.

As indicated, in one embodiment, the medical event processing computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the medical event processing computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the medical event processing computing entity 106 may include or be in communication with one or more input elements, such as a keyboard input, a mouse input, a touch screen/display input, motion input, movement input, audio input, pointing device input, joystick input, keypad input, and/or the like. The medical event processing computing entity 106 may also include or be in communication with one or more output elements (not shown), such as audio output, video output, screen/display output, motion output, movement output, and/or the like.

### Exemplary Patient Computing Entity

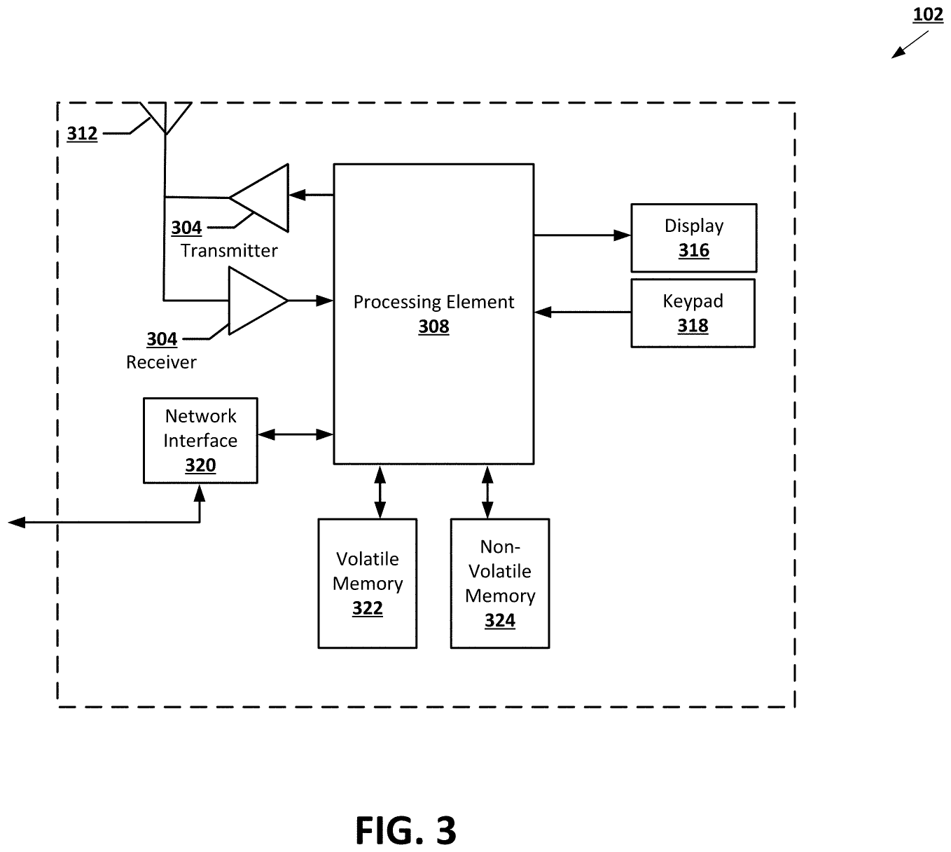

FIG. 3 provides an illustrative schematic representative of a patient computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Patient computing entities 102 can be operated by various parties. As shown in FIG. 3, the patient computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the patient computing entity 102 may be capable of operating with one or more air interface standards, communication protocols, modulation types, and access types. More particularly, the patient computing entity 102 may operate in accordance with any of a number of wireless communication standards and protocols, such as those described above with regard to the medical event processing computing entity 106. In a particular embodiment, the patient computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the patient computing entity 102 may operate in accordance with multiple wired communication standards and protocols, such as those described above with regard to the medical event processing computing entity 106 via a network interface 320.

Via these communication standards and protocols, the patient computing entity 102 can communicate with various other entities using concepts such as Unstructured Supplementary Service Data (USSD), Short Message Service (SMS), Multimedia Messaging Service (MIMS), Dual-Tone Multi-Frequency Signaling (DTMF), and/or Subscriber Identity Module Dialer (SIM dialer). The patient computing entity 102 can also download changes, add-ons, and updates, for instance, to its firmware, software (e.g., including executable instructions, applications, program modules), and operating system.

According to one embodiment, the patient computing entity 102 may include location determining aspects, devices, modules, functionalities, and/or similar words used herein interchangeably. For example, the patient computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the patient computing entity's 102 position in connection with a variety of other systems, including cellular towers, Wi-Fi access points, and/or the like. Similarly, the patient computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The patient computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the patient computing entity 102 to interact with and/or cause display of information/data from the medical event processing computing entity 106, as described herein. The user input interface can comprise any of a number of devices or interfaces allowing the patient computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the patient computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The patient computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the patient computing entity 102. As indicated, this may include a user application that is resident on the entity or accessible through a browser or other user interface for communicating with the medical event processing computing entity 106 and/or various other computing entities.

In another embodiment, the patient computing entity 102 may include one or more components or functionality that are the same or similar to those of the medical event processing computing entity 106, as described in greater detail above. As will be recognized, these architectures and descriptions are provided for exemplary purposes only and are not limiting to the various embodiments.

In various embodiments, the patient computing entity 102 may be embodied as an artificial intelligence (AI) computing entity, such as an Amazon Echo, Amazon Echo Dot, Amazon Show, Google Home, and/or the like. Accordingly, the patient computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### Exemplary Provider Computing Entity

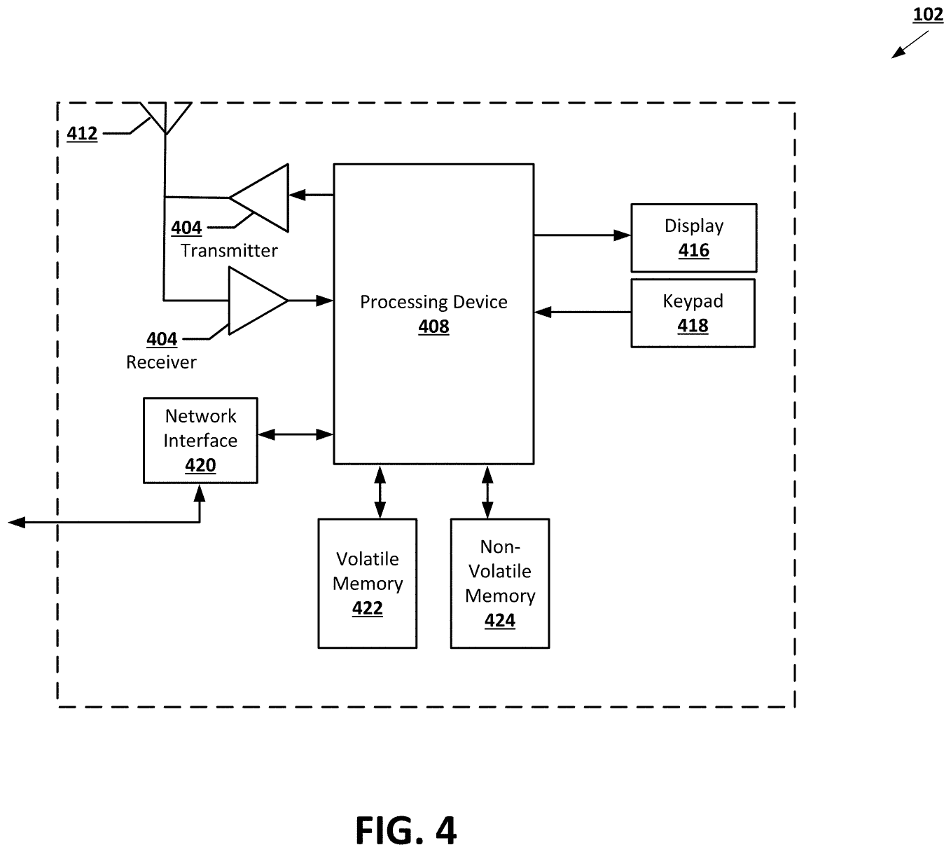

FIG. 4 provides an illustrative schematic representative of a provider computing entity 103 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Provider computing entities 103 can be operated by various parties. As shown in FIG. 4, the provider computing entity 103 can include an antenna 412, a transmitter 404 (e.g., radio), a receiver 406 (e.g., radio), and a processing element 408 (e.g., CPLDs, microprocessors, multi-core processors, coprocessing entities, ASIPs, microcontrollers, and/or controllers) that provides signals to and receives signals from the transmitter 404 and receiver 406, correspondingly.

The signals provided to and received from the transmitter 404 and the receiver 406, correspondingly, may include signaling information/data in accordance with air interface standards of applicable wireless systems. In this regard, the provider computing entity 103 may be capable of operating with one or more air interface standards, communication protocols, modulation types, and access types. More particularly, the provider computing entity 103 may operate in accordance with any of a number of wireless communication standards and protocols, such as those described above with regard to the medical event processing computing entity 106. In a particular embodiment, the provider computing entity 103 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the provider computing entity 103 may operate in accordance with multiple wired communication standards and protocols, such as those described above with regard to the medical event processing computing entity 106 via a network interface 420.

Via these communication standards and protocols, the provider computing entity 103 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The provider computing entity 103 can also download changes, add-ons, and updates, for instance, to its firmware, software (e.g., including executable instructions, applications, program modules), and operating system.

According to one embodiment, the provider computing entity 103 may include location determining aspects, devices, modules, functionalities, and/or similar words used herein interchangeably. For example, the provider computing entity 103 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the provider computing entity's 103 position in connection with a variety of other systems, including cellular towers, Wi-Fi access points, and/or the like. Similarly, the provider computing entity 103 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The provider computing entity 103 may also comprise a user interface (that can include a display 416 coupled to a processing element 408) and/or a user input interface (coupled to a processing element 408). For example, the user interface may be a user application, browser, user interface, and/or similar words used herein interchangeably executing on and/or accessible via the provider computing entity 103 to interact with and/or cause display of information/data from the medical event processing computing entity 106, as described herein. The user input interface can comprise any of a number of devices or interfaces allowing the provider computing entity 103 to receive data, such as a keypad 418 (hard or soft), a touch display, voice/speech or motion interfaces, or other input device. In embodiments including a keypad 418, the keypad 418 can include (or cause display of) the conventional numeric (0-9) and related keys (#, *), and other keys used for operating the provider computing entity 103 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The provider computing entity 103 can also include volatile storage or memory 422 and/or non-volatile storage or memory 424, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the provider computing entity 103. As indicated, this may include a user application that is resident on the entity or accessible through a browser or other user interface for communicating with the medical event processing computing entity 106 and/or various other computing entities.

In another embodiment, the provider computing entity 103 may include one or more components or functionality that are the same or similar to those of the medical event processing computing entity 106, as described in greater detail above. As will be recognized, these architectures and descriptions are provided for exemplary purposes only and are not limiting to the various embodiments.

In various embodiments, the provider computing entity 103 may be embodied as an artificial intelligence (AI) computing entity, such as an Amazon Echo, Amazon Echo Dot, Amazon Show, Google Home, and/or the like. Accordingly, the provider computing entity 103 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### IV. EXEMPLARY SYSTEM OPERATIONS

To address the noted technical challenges associated with efficiency and reliability of medical event processing systems, various embodiments of the present invention disclose performing/executing temporally dynamic medical event processing. In one aspect of performing/executing temporally dynamic medical event processing relates to reducing the need for real-time processing of patient data and provider data in determining patient needs and operational readiness of available providers to address the detected patient needs. Of note, various embodiments of the temporally dynamic medical event processing concepts accomplish a reduction in real-time processing need without ignoring temporal properties of medical need scenarios. In doing so, the noted embodiments reduce computational complexity of medical event processing routines without comprising accuracy of such routines to the extent such accuracy is a result of integrating temporal properties of medical need scenarios into predictive inferences.

For example, according to some embodiments, a temporally dynamic medical event processing system generates temporally dynamic service quality models for service providers based in part on static data determined entirely based at least in part on relating historic operational trends of the service providers to static properties of a time interval associated with a medical need scenario (e.g., based at least in part on time-of-day, date-of-week, and/or the like). Such static generation of service quality models decreases the need for real-time processing to generate service quality models without ignoring temporally unique properties of medical need scenarios. As another example, according to some embodiments, a temporally dynamic medical event processing system generates temporally dynamic service quality models for service providers based in part on static data determined entirely based at least in part on relating historic operational trends of the service providers to dynamic properties of a time interval associated with a medical need scenario, where the dynamic properties are determined based at least in part on current operational trends of the service providers. Such utilization of historic operational data in processing current operational trends provides another mechanism for decreasing the need for real-time processing to generate service quality models without ignoring temporally unique properties of medical need scenarios.

Using the above-described techniques and related techniques described throughout this document, various embodiments of the temporally dynamic medical event processing concepts accomplish a reduction in real-time processing need without ignoring temporal properties of medical need scenarios.

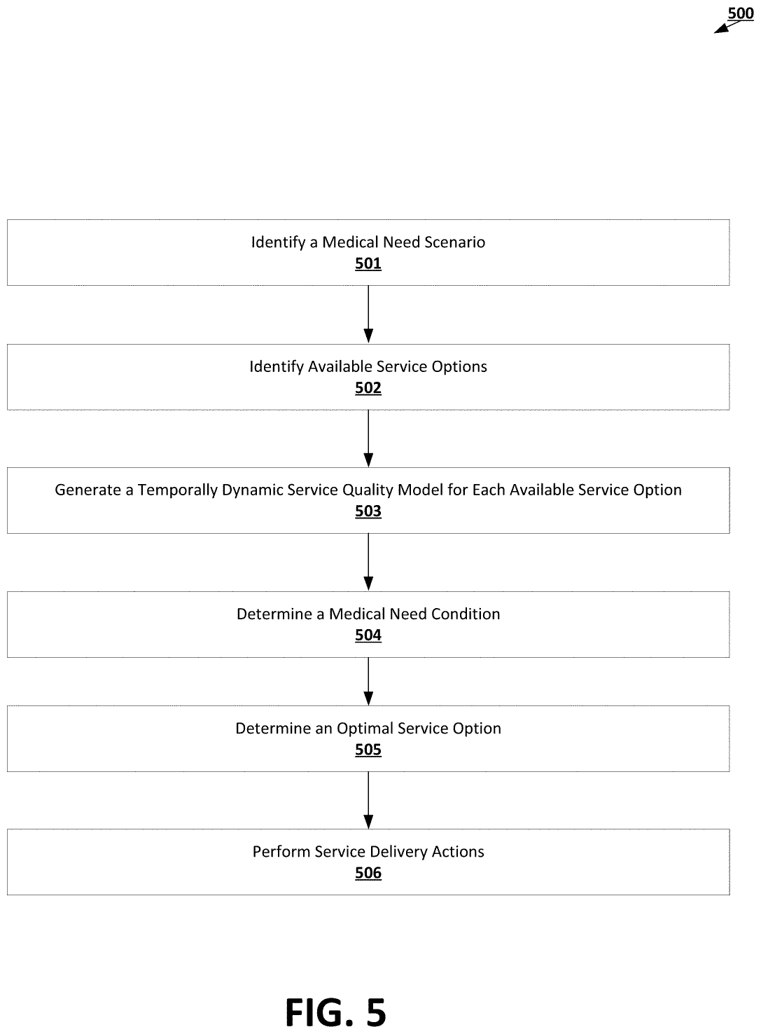

FIG. 5 is a flowchart diagram of an example process 500 for temporally dynamic medical event processing. Via the various steps/operations of process 500, the medical event processing computing entity 106 can utilize data obtained from patient computing entities 102 and from provider computing entities 103 as well as historical data such as historical patient health data and historical provider performance data to perform predictive inferences configured to estimate medical and provider response readiness and perform prediction-based actions in an efficient and effective manner.

The process 500 begins at step/operation 501 when the medical condition modeling engine 112 of the medical event processing computing entity 106 identifies a medical need scenario associated with a patient profile. In some embodiments, a medical scenario is a data object that characterizes a set of recorded real-world observations associated with a patient profile as potentially indicating a need for medical response. In some embodiments, the medical need scenario includes underlying data describing the characterized set of real-world observations.

In some embodiment, the characterized set of real-world observations include observations determined based at least in part on the receipt of at least one of directed input data and undirected input data by the medical event processing computing entity 106. Directed input data may include information entered by an end user to an end user computing entity, e.g., information entered by a patient end user associated with a patient profile to a patient computing entity 102 associated with the patient profile. Examples of directed input data may include message data authored and sent by an end user, information entered by an end user into one or more applications (e.g., addresses provided to a navigation application and voice prompts provided to a voice assistant application), and/or the like. Undirected input data may include information obtained by monitoring conditions of an end user and/or conditions of an environment of an end user, where the noted information items are not entered by the end user. Examples of undirected input data include location data (e.g., Global Positioning System (GPS) coordinates of an end user over time), information obtained by monitoring voice conversations of an end user, information obtained by monitoring one or more video feeds associated with an environment of an end user, information obtained by monitoring network connections of an end user, and/or the like.

At step/operation 502, the temporal service modeling engine 111 of medical event processing computing entity 106 identifies one or more available service options for the medical need scenario. In some embodiments, the temporal service modeling engine 111 selects available service options for the medical need scenario based at least in part on estimated travel times between a set of candidate available service option facilities and a geographic region associated with the medical need scenario. In some embodiments, the temporal service modeling engine 111 determines travel times between service option facilities and particular geographical regions based at least in part on historic traffic data and historical ambulance dispatch speed data associated with the medical providers.

In some embodiments, to identify available service options for a medical need scenario, the temporal service modeling engine 111 retrieves location data associated with the medical need scenario, determines a service region of the medical need scenario based at least in part on the retrieved location data, and queries a provider database stored in the storage subsystem 108 for available medical providers that are within a threshold travel time from the service region. In some embodiments, to identify available service options for the medical need scenario, the temporal service modeling engine 111 retrieves location data associated with the medical need scenario, determines a service region of the medical need scenario based at least in part on the retrieved location data, and queries a provider database stored in the storage subsystem 108 for a threshold numbers of available medical providers that are deemed to have the lowest travel times from the service region. In some embodiments, to identify available service options for the medical need scenario, the temporal service modeling engine 111 retrieves location data associated with the medical need scenario, determines a service region of the medical need scenario based at least in part on the retrieved location data, queries a provider database stored in the storage subsystem 108 for a first set of available medical providers that are within a threshold travel time from the service region, and selects a threshold number of the first set of available medical providers based at least in part on the travel times from the service region to each of the first set of available medical providers.

At step/operation 503, the temporal service modeling engine 111 generates a temporally dynamic service quality model for each available service option identified in step/operation 502. In some embodiments, a temporally dynamic service quality model for an available service option is a data object that indicates a current estimated service quality score for the available service option with respect to each candidate medical need condition of one or more candidate medical need conditions associated with the medical event processing computing entity 106 based at least in part on historic operational data associated with operation of the available service option as well as current (e.g., real-time) operational data associated with operation of the available service option. For example, a temporally dynamic service quality model for a particular hospital may indicate a current estimated service quality score for the particular hospital with respect to handling gunshot wounds, a current estimated service quality score for the particular hospital with respect to handling seizure patients, a current estimated service quality score for the particular hospital with respect to handling alcohol poisoning, and/or the like. In some embodiments, at least one of the noted current estimated service quality scores may be determined based at least in part on historic operational data associated with operation of the hospital as well as current operational data associated with operation of the particular hospital.

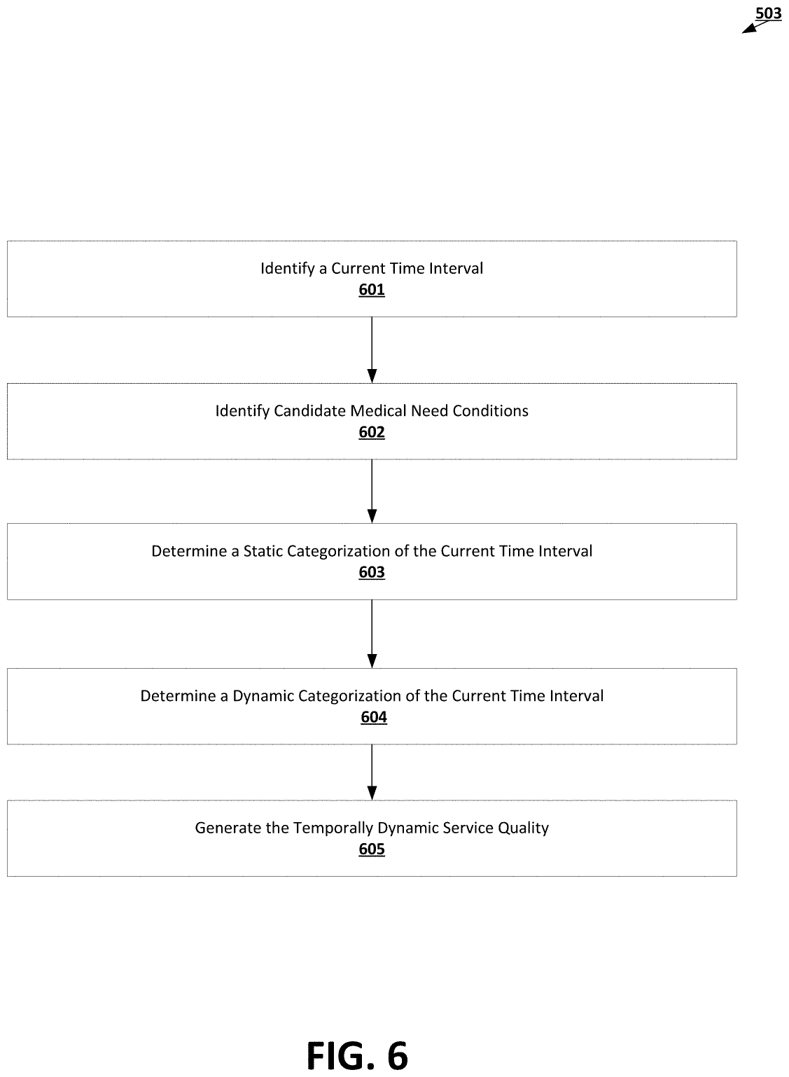

In some embodiments, generating a temporally dynamic service quality model for a particular available service option may be performed in accordance with the process depicted in FIG. 6. The process depicted in FIG. 6 begins at step/operation 601 when the temporal service modeling engine 111 identifies a current time interval associated with the medical need scenario. At step/operation 602, the temporal service modeling engine 111 identifies one or more candidate medical need conditions associated with the patient response computing entity 106. In some embodiments, each candidate medical need condition is characterized by at least one of a patient competence expectation, a patient mobility expectation, and a patient health expectation. For example, a particular candidate medical need condition may describe condition of patients who are competent, can transport themselves to a medical facility, and suffer from non-lethal wounds. As another example, a particular candidate medical need condition may describe condition of wounded patients regardless of their competence and mobility capabilities.

At step/operation 603, the temporal service modeling engine 111 determines a static categorization of the current time interval. At step/operation 604, the temporal service modeling engine 111 determines a dynamic categorization of the current time interval. In some embodiments, the static categorization of the current time interval is a data object that characterizes the current time interval based at least in part on prior (e.g., previously known) properties of the current time interval and/or prior properties of the particular available service option during the current time interval, while the dynamic categorization of the current time interval is a data object that characterizes the current time interval based at least in part on data received about a current operational state of the particular available service option and/or a current operational state of an environment of the particular available service option. An example of a static categorization of a current time interval is a characterization of the current time interval based at least in part on at least one of date of the current time interval, time-of-day of the current time interval, a general frequency of a particular health condition at the current time interval (e.g., characterization of weekend nights as likely associated with higher instances of alcohol poisoning), a general availability of doctors at the particular service option at the current time interval, and/or the like. An example of a dynamic categorization of a current time interval is a characterization of the current time interval based at least in part on current available doctor data for the particular available service option, current traffic data in a region of the particular available service option, current wait time for the particular available service option.

In some embodiments, the static categorization of the particular time interval is determined based at least in part on facility capability data associated with the particular available service option. Facility capability data may include such as data about whether emergency power generators for particular service option facility are antiquated, undersized, regularly maintained, regularly tested and approved, utilize effective time analysis, and/or the like; data about fuel dependencies of the particular service option facility; data about fuel delivery methods and fuel delivery availabilities of the particular service option facility; data about dual power entrance capabilities of the particular service option facility; and/or the like. In some embodiments, to determine the facility capability data, the temporal service modeling engine 111 performs at least one of the following sets of operations: (i) obtaining (e.g., from a time tracking system and/or a human resources management system) employee hours dedicated to generator maintenance and repair and determining whether the noted employee hours satisfy a threshold (e.g., one hour); (ii) obtaining (e.g., from a transactional record maintenance system) purchase history data associated with purchase of generators, purchase of generator parts, purchase of generator maintenance services, fuel purchases, and/or the like, utilizing the obtained purchase history data to determine a measure of health and fuel need condition of the generator, and determining whether the noted measure exceeds a threshold; (iii) obtaining (e.g., from a transactional record maintenance system) supplier location data associated with current fuel suppliers for the facility, determining a travel time from supplier locations to the facility, and determining whether the travel time satisfies a threshold; and (iv) determining a facility rating for the facility based at least in part on nationally known standards and local competitive facility standards.

In some embodiments, the static categorization of the particular time interval is determined based at least in part on emergency response capability data of the particular available service option. Examples of the emergency response capability data of the particular available service option include data about at least one of whether the particular available service option partners with volunteer organizations such as volunteer ham radio organizations or local emergency operation centers, whether the particular available service option has done a sufficiently recent emergency operations coordinated testing, whether the particular available service option maintains a business community plan for times of emergency, and past success of the particular available service option in addressing emergency situations. In some embodiments, to determine the emergency response capability data of the particular available service option, the temporal service modeling engine 111 performs at least one of the following sets of operations: (i) processing employee hour records and communication log records to determine a measure of availability of staff and other response capabilities during emergency time intervals and determining whether the noted measure of availability satisfies a threshold; (ii) processing emergency readiness certification data, emergency readiness rating data, and business continuity data to determine a measure of emergency readiness and determine whether the noted measure of emergency readiness satisfies a threshold; (iii) processing cell tower coverage data for the particular service option facility and determining whether the cell tower coverage for the particular service option facility satisfies a threshold.

In some embodiments, the static categorization of the particular time interval is determined based at least in part on medication availability data of the particular available service option. Examples of medication availability data of the particular available service option include whether the particular available service option facility has an onsite pharmacy, whether the particular available service option facility uses an automated drug delivery system, whether the medication availability capabilities of particular available service option facility have passed accountability audits in the past, and/or the like. In some embodiments, to determine medication availability data of the particular available service option, the temporal service modeling engine 111 may utilize pharmacy data as well as purchase data associated with purchase of medications.

Other examples of the static categorization of the particular time interval is determined based at least in part on at least one of the following: (i) data about total bed counts of the particular available service option, (ii) data about expected number of available service providers (e.g., doctors, nurses, and/or the like) at the particular available service option during the time interval based at least in part on shift scheduling data associated with the particular available service option, (iii) data about whether the particular available service option is accessible by ambulances and/or helicopters, (iv) data about any ratings of the particular available service option by independent organizations, (v) data about financial state of the particular available service option, (vi) data about age of the particular available service option facility, (vii) data about whether the particular available service option has state of the art equipment, and (viii) data about health outcomes of the particular service option (e.g., mortality rates, malpractice lawsuits, secondary infection rates, return visit rates, and/or the like).

In some embodiments, the dynamic categorization of the particular time interval is determined based at least in part on a current specialist availability for the particular service option, a current medication quantity availability for the particular service optio, a current measure of wait time at the particular service option (e.g., a measure of wait time determined based at least in part on video feedback data), current environmental conditions at the particular service option facility determined based at least in part on one or more environmental condition signals received from one or more Internet of Things (TOT) computing entities installed at the particular service option facility, and/or the like.

At step/operation 605, the temporal service modeling engine 111 generates the temporally dynamic service quality model for the particular available service option based at least in part on the static categorization of the current time interval determined at step/operation 604 and dynamic categorization of the current time interval determined at step/operation 605. In some embodiments, to determine the temporally dynamic service quality model for the particular available service option, the temporal service modeling engine 111 estimates a current estimated service quality score for the particular available service option with respect to each candidate medical need condition identified at step/operation 602 by determining whether the static categorization of the current time interval and dynamic categorization of the current time interval correspond to one or more medical response need expectations of each candidate medical need condition (e.g., a patient competence expectation of a candidate medical need condition, a patient mobility expectation of a candidate medical need condition, a patient health expectation of a candidate medical need condition, and/or the like).

For example, if the dynamic categorization of the current interval indicates a low number of available beds at the particular available service option, the temporal service modeling engine 111 may generate a temporally dynamic service quality model that includes a low current estimated service quality score for the particular available service option with respect to any candidate medical need condition that requires a bed. As another example, if the dynamic categorization of the current interval indicates a high number of available beds at the particular available surgeons, the temporal service modeling engine 111 may generate a temporally dynamic service quality model that includes a high current estimated service quality score for the particular available service option with respect to any candidate medical need condition that requires surgery. As a further example, if the dynamic categorization of the current interval indicates a high number of available ambulances at the particular available surgeons (e.g., in the case of an unconscious, impatient, and/or immobile patient), the temporal service modeling engine 111 may generate a temporally dynamic service quality model that includes a high current estimated service quality score for the particular available service option with respect to any candidate medical need condition that requires ambulance transportation.

Returning to FIG. 5, at step/operation 504, the medical conditioning modeling engine 112 determines a medical need condition of the patient profile. In some embodiments, to determine a medical need condition of the patient profile, the medical conditioning modeling engine 112 selects a particular candidate medical need condition from the one or more candidate medical need conditions associated with the medical event processing computing entity 106 based at least in part on at least one of the directed input data and the undirected input data introduced above. In some embodiments, the medical conditioning modeling engine 112 receives at least one directed input and undirected input data from one or more IOT devices associated with monitoring conditions of the patient associated with the patient profile and/or conditions of an environment of the patient associated with the patient profile.

In some embodiments, to obtain directed input data, the medical conditioning modeling engine 112 generates a prompt to a patient profile in response to identifying the medical need scenario at step/operation 501, receives user-entered data in response to the generated prompt, and determines the directed input data based at least in part on the received user-entered data. In some embodiments, if the medical conditioning modeling engine 112 does not receive user-entered data in response to the generated prompt, the medical conditioning modeling engine 112 determines that the patient associated with the patient profile may be unconscious and/or incompetent. In some embodiments, the medical conditioning modeling engine 112 generates the prompt based at least in part on audible and/or visual properties configured to accommodate to one or more known health conditions of the patient associated with the patient profile. For example, the medical conditioning modeling engine 112 may generate a loud audible noise as part of a notification to the patient end user if the medical conditioning modeling engine 112 determines (e.g., based at least in part on patient medical history data and/or based at least in part on accessibility settings of the patient computing device 102 associated with the patient) that the patient end user suffers from hearing issues.

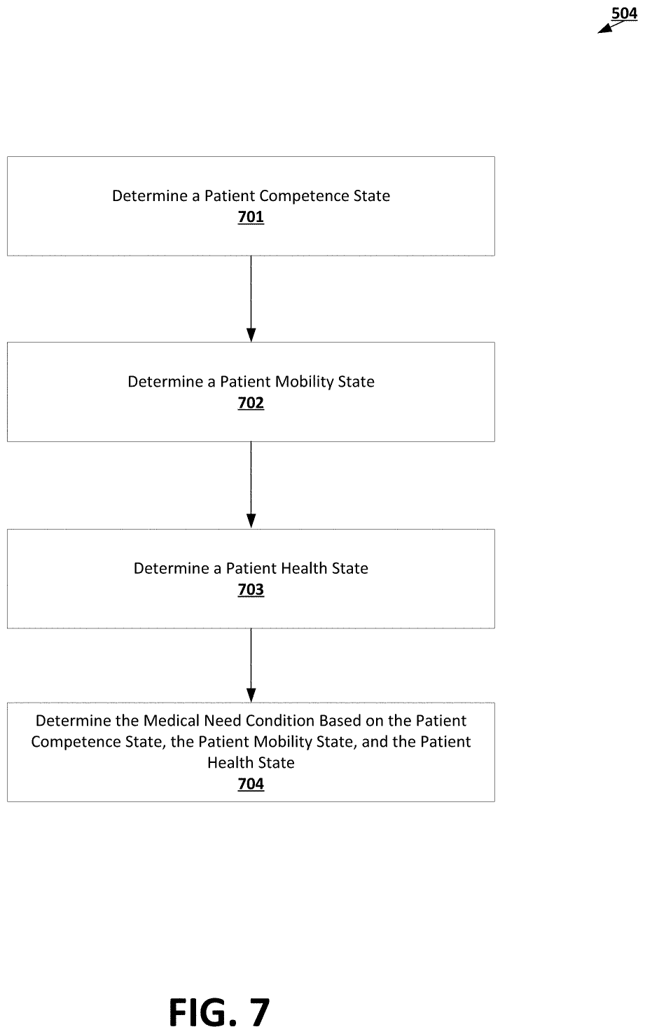

In some embodiments, step/operation 504 may be performed in accordance with the process depicted in FIG. 7. The process depicted in FIG. 7 begins at step/operation 701 when the medical conditioning modeling engine 112 determines a patient competence state of the patient profile. The patient competence state of the patient profile is a determination about whether a patient associated with the patient profile can communicate information about the patient's health conditions. The medical conditioning modeling engine 112 may determine the patient competence state of the patient profile based at least in part on at least one of the directed input data and the undirected input data introduced above. For example, the medical conditioning modeling engine 112 may determine that the patient profile is incompetent if the medical conditioning modeling engine 112 does not receive any directed input data from the patient profile and/or if the undirected input data received from an environment of the patient profile indicates that the patient is incompetent (e.g., data indicates no movement within a threshold amount of time). In some embodiments, medical conditioning modeling engine 112 may determine the patient competence state of the patient profile at least in part based at least in part on historical health data associated with the patient profile.

At step/operation 702, the medical conditioning modeling engine 112 determines a patient mobility state of the patient profile. The patient competence state of the patient profile is a determination about whether a patient associated with the patient profile requires assistance to travel to a medical facility. The medical conditioning modeling engine 112 may determine the patient mobility state of the patient profile based at least in part on at least one of the directed input data and the undirected input data introduced above. For example, the medical conditioning modeling engine 112 may determine that the patient associated with the patient profile is mobile if the patient enters a direction into a navigation application, if the patient appears to be walking in a video feed, or if the patient appears to utter sentences like “I am on the way to the hospital.” In some embodiments, medical conditioning modeling engine 112 may determine the patient mobility state of the patient profile at least in part based at least in part on historical health data associated with the patient profile. For example, if the patient associated with the patient profile is known to suffer from a walking disability, the medical conditioning modeling engine 112 may determine that the patient profile is immobile.

At step/operation 703, the medical conditioning modeling engine 112 determines a patient health state of the patient profile. The patient health state of the patient profile is a determination about the nature and/or severity of a current medical need of a patient associated with the patient. The medical conditioning modeling engine 112 may determine the patient health state of the patient profile based at least in part on at least one of the directed input data and the undirected input data introduced above. For example, the medical conditioning modeling engine 112 may determine that the patient associated with the patient profile suffers from a gunshot wound based at least in part on monitoring conversations of the patient indicating occurrence of a recent gunshot wound. As another example, the medical conditioning modeling engine 112 may determine that the patient associated with the patient profile suffers from a heart condition based at least in part on monitoring pulse of the patient using sensory data transmitted by the patient computing entity 102 associated with the patient profile. In some embodiments, medical conditioning modeling engine 112 may determine the patient health state of the patient profile at least in part based at least in part on historical health data associated with the patient profile. For example, if the patient associated with the patient profile is known to suffer from occasional seizures, the medical conditioning modeling engine 112 may determine that the patient has likely suffered from a seizure in response to determining that the patient profile has an unconscious state.

At step/operation 704, the medical conditioning modeling engine 112 determines the medical need condition based at least in part on the patient competence state determined at step/operation 701, the patient mobility state determined at step/operation 702, and the patient health state determined at step/operation 703. In some embodiments, the medical need condition is a multi-dimensional vector, where each value of the vector represents an aspect of a condition of the patient profile and/or a condition of the environment of the patient profile. In some embodiments, at least three values of the multi-dimensional vector relate to the patient competence state, the patient mobility state, and the patient health state respectively. In some embodiments, to determine a medical need condition of the patient profile, the medical conditioning modeling engine 112 selects a particular candidate medical need condition from the one or more candidate medical need conditions associated with the medical event processing computing entity 106 based at least in part on at least one of the patient competence state, the patient mobility state, and the patient health state.

Returning to FIG. 5, at step/operation 505, the temporal service modeling engine 111 determines an optimal service option from the available service options identified in step/operation 502 based at least in part on each temporally dynamic service quality model generated in step/operation 503 and the medical need condition determined in step/operation 504. In some embodiments, to determine the optimal service option, the temporal service modeling engine 111 retrieves, from each temporally dynamic service quality model for an available service option, the current estimated service quality score for the available service option with respect to the medical need condition. The temporal service modeling engine 111 then selects the available service option having the highest current estimated service quality score as the optimal service option. In some embodiments, if the patient mobility state of the medical need condition indicates an immobile patient and/or an incompetent state (e.g., an unconscious state), the medical conditioning modeling engine 112 determines a relatively low current estimated service quality score (e.g., a minimal score, such as score of zero) for an available service option with respect to the medical need condition if the available service option does not have ambulance and/or helicopter transport capabilities. In some embodiments, the temporal service modeling engine 111 performs assignment of patients to medical facilities based at least in part on mobility and/or emergency response capabilities of the medical facilities.

At step/operation 506, the temporal service modeling engine 111 performs one or more service delivery actions in relation to at least one of the patient profile and the optimal service option. In some embodiments, the temporal service modeling engine 111 notifies a patient computing entity 102 associated with the patient profile of a recommendation of the optimal service option. In some embodiments, the temporal service modeling engine 111 notifies a provider computing entity 103 of the of the recommendation of the optimal service option to a particular patient profile having particular properties and a particular detected medical need condition. In some embodiments, the temporal service modeling engine 111 automatically schedules an appointment, an ambulance arrangement, and/or a ridesharing arrangement for transferring the patient associated with the patient profile to the optimal service option facility.

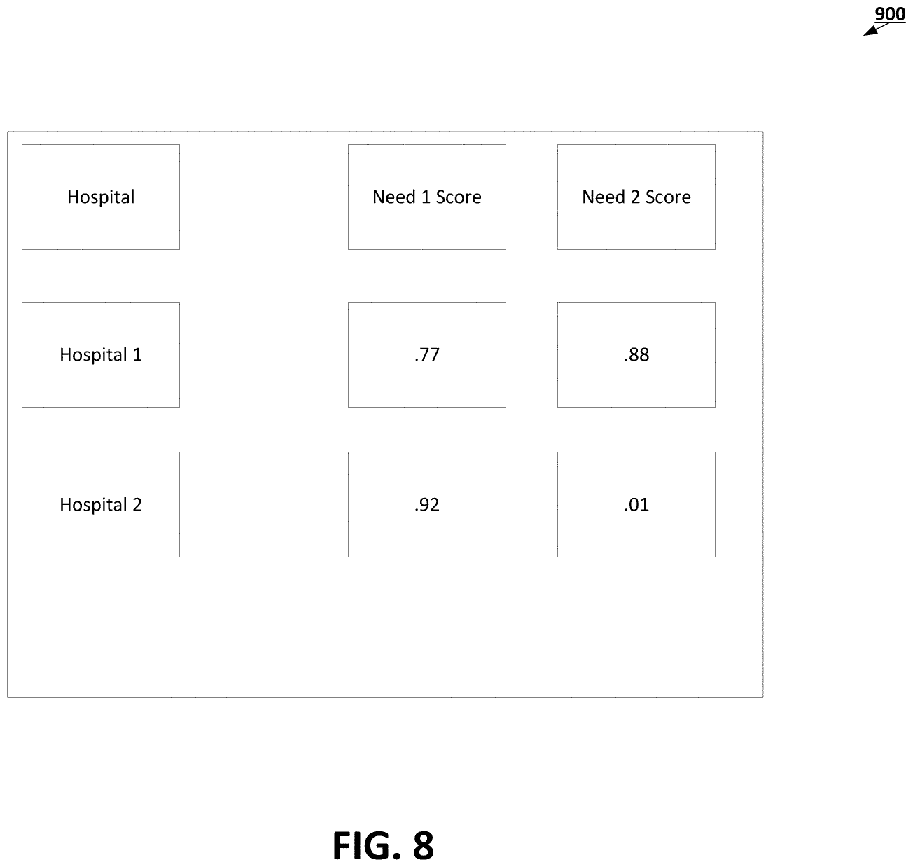

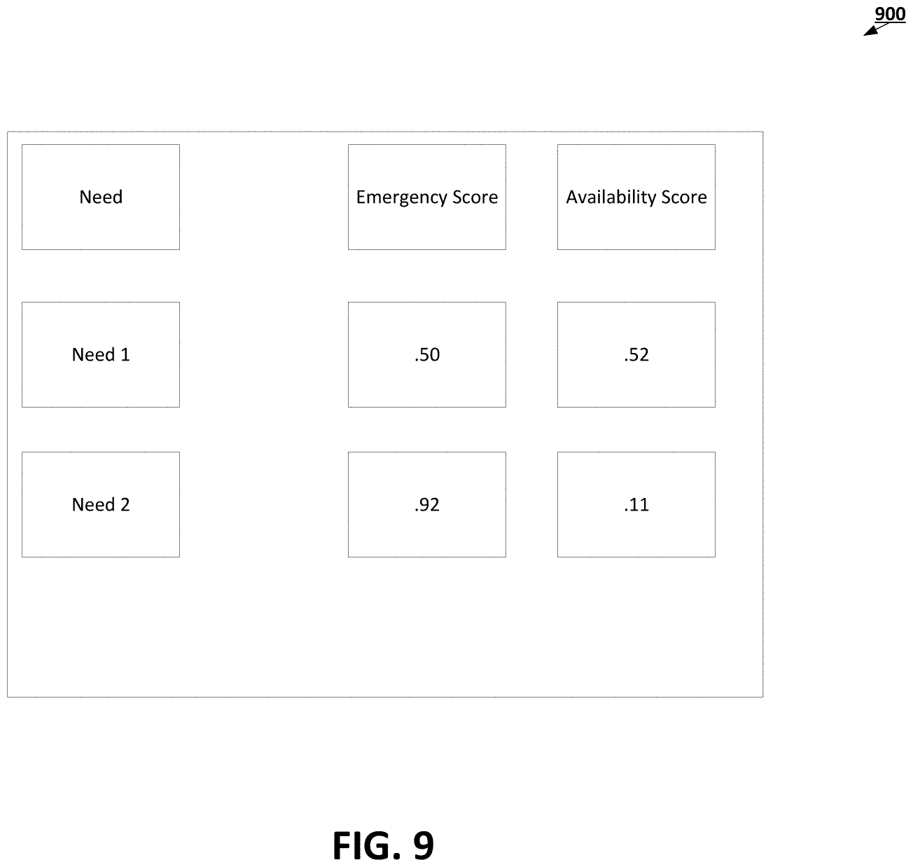

In some embodiments, the temporal service modeling engine 111 notifies an operational management unit and/or a load balancing unit of the provider computing entity 103 of the optimal service option about operational readiness of the optimal service option in relation to other service options, e.g., using a provider operational readiness user interface configured to display relative operational readiness of one or more available service options. An example of a provider operational readiness user interface 800 is depicted in FIG. 8. In some embodiments, the temporal service modeling engine 111 notifies the patient computing entity 102 of the patient profile about medical need service availability of the detected medical needs of the patient profile, using a medical need service availability user interface configured to display relative operational readiness of one or more available service options to handle the detected medical needs of the patient profile. An example of a medical need service availability user interface 900 is depicted in FIG. 9.

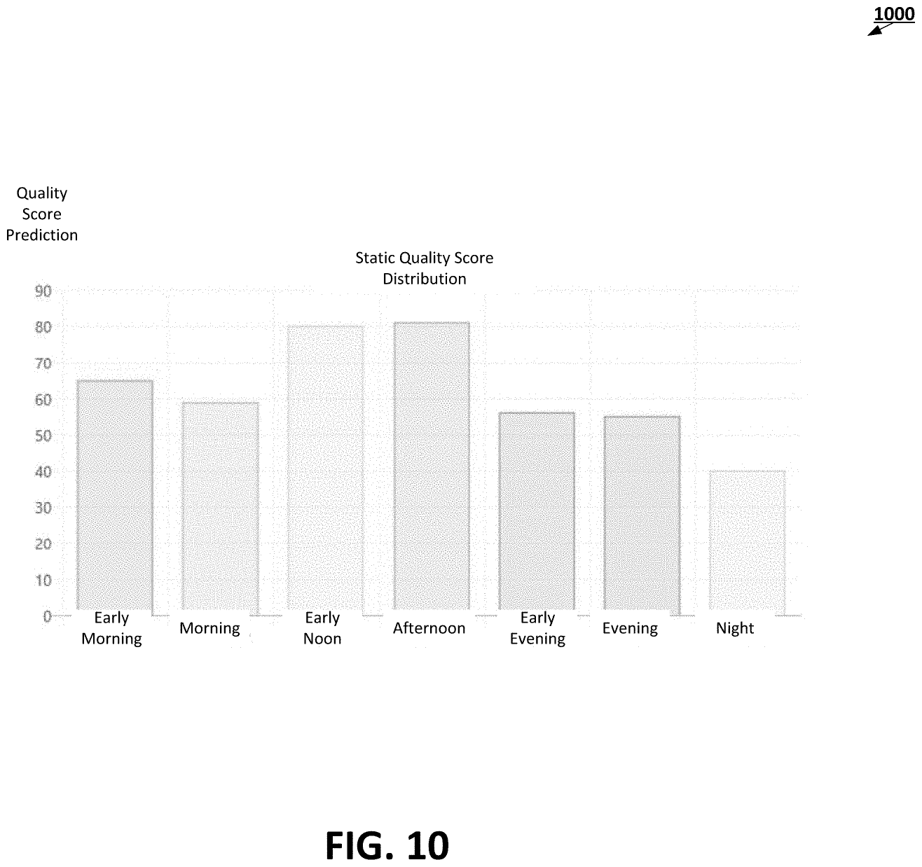

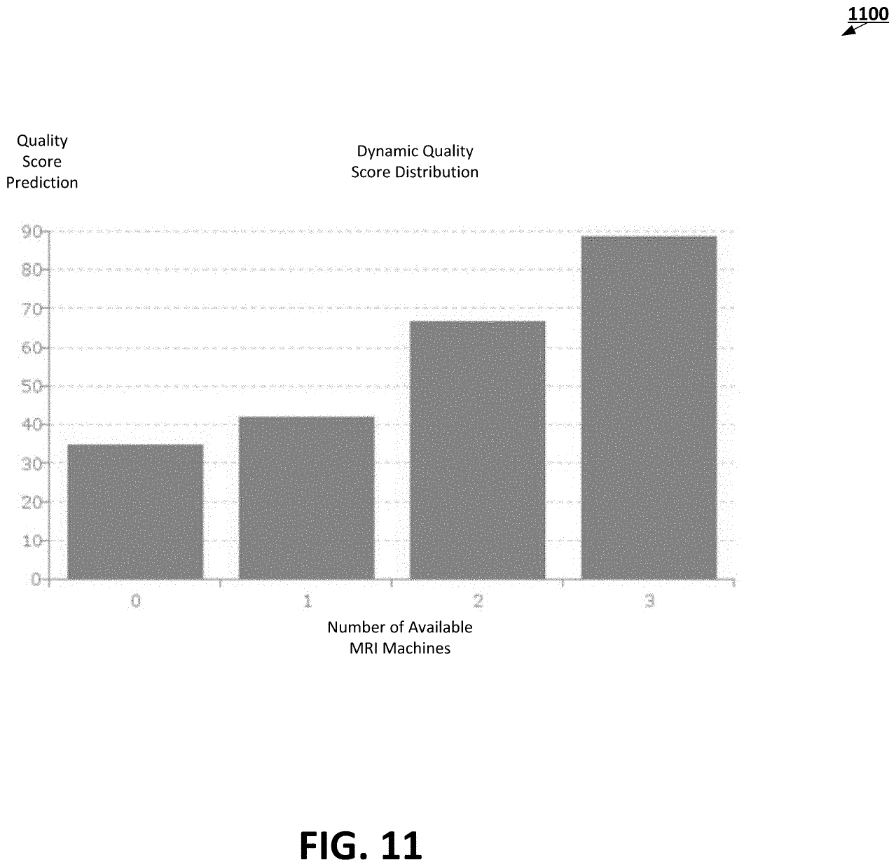

In some embodiments, the temporal service modeling engine 111 notifies an operational management unit and/or a load balancing unit of the provider computing entity 103 of the optimal service option about operational readiness of the optimal service option to handle one or more designated medical needs during various statically defined time intervals using a static quality score distribution user interface, such as the static quality score distribution user interface 1000 of FIG. 10. In some embodiments, the temporal service modeling engine 111 notifies an operational management unit and/or a load balancing unit of the provider computing entity 103 of the optimal service option about operational readiness of the optimal service option to handle one or more designated medical needs during various dynamically defined time intervals using a dynamic quality score distribution user interface, such as the dynamic quality score distribution user interface 1100 of FIG. 11.

### V. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing temporally dynamic medical event processing with respect to a medical need scenario, the computer-implemented method comprising:
determining, by one or more processors, a medical need condition of a patient profile associated with the medical need scenario, wherein (i) the medical need condition is selected from a plurality of candidate medical need conditions, (ii) each of the plurality of candidate medical need conditions is characterized by one or more medical response expectations comprising a patient competence expectation, and (iii) determining the medical need condition is based at least in part on a patient competence state associated with the medical need scenario;
determining, by the one or more processors, a temporally dynamic service quality model for each available service option of a plurality of available service options associated with the patient profile, wherein: (i) the corresponding temporally dynamic service quality model for an available service option indicates a current estimated service quality score for the available service option with respect to each candidate medical need condition of the plurality of candidate medical need conditions, and (ii) determining the temporally dynamic service quality model for an available service option comprises assigning a minimal current estimated service quality score for the available service option with respect to each candidate medical need condition of a plurality of candidate medical need conditions in response to determining that a transport capability designation of the available service option does not satisfy the patient competence expectation of the candidate medical need condition;
determining, by the one or more processors and based at least in part on the medical need condition and each temporally dynamic service quality model for an available service option of the plurality of available service options, an optimal service option of the plurality of available service options; and
performing, by the one or more processors, one or more service delivery actions by communicating with at least one of a patient computing entity associated with the patient profile or a provider computing entity associated with the optimal service option.

2. The computer-implemented method of claim 1, wherein:
each of the one or more medical response expectations for a candidate medical need condition of the plurality of candidate medical need conditions is characterized by a patient mobility expectation for the candidate medical need condition,
determining the medical need condition is based at least in part on a patient mobility state associated with the medical need scenario, and
determining the temporally dynamic service quality model for an available service option of the plurality of available service options comprises assigning a minimal current estimated service quality for the available service option with respect to each candidate medical need condition of a plurality of candidate medical need conditions in response to determining that the transport capability designation of the available service option does not satisfy the patient mobility expectation of the candidate medical need condition.

3. The computer-implemented of claim 1, wherein:
each of the one or more medical response expectations for a candidate medical need condition of the plurality of candidate medical need conditions is characterized by a patient health expectation for the candidate medical need condition,
determining the medical need condition is based at least in part on a patient health state associated with the medical need scenario, and
determining the temporally dynamic service quality model for an available service option of the plurality of available service options comprises assigning a minimal current estimated service quality for the available service option with respect to each candidate medical need condition of a plurality of candidate medical need conditions in response to determining that a service capability designation of the available service option does not satisfy the patient health expectation of the candidate medical need condition.

4. The computer-implemented method of claim 1, wherein each current estimated service quality score indicated by a temporally dynamic service quality model for an available service option of the plurality of available service options is determined based at least in part on historic operational data associated with the available service option and real-time operational data associated with the available service option.

5. The computer-implemented method of claim 4, wherein determining the temporally dynamic service quality model for an available service option of the plurality of available service options comprises:
identifying a current time interval associated with the medical need scenario and the available service option;
determining, based at least in part on the historic operational data, a static characterization of the current time interval based at least in part on one or more historic operational trends of the available service option with respect to one or more static properties of the available service option during the current time interval;
determining, based at least in part on the real-time operational data, a dynamic characterization of the current time interval based at least in part on one or more current operational trends of the available service option;
determining, based at least in part on the static characterization of the current time interval and the dynamic characterization of the current time interval, a temporal designation of the current time interval; and
determining the current estimated service quality score for the available service option with respect to each candidate medical need condition of the plurality of candidate medical need conditions based at least in part on comparing the temporal designation of the current time interval to one or more medical response expectations of the medical need condition.

6. The computer-implemented method of claim 5, wherein the one or more static properties comprise a time-of-day property and a day-of-week property.

7. The current-implemented method of claim 5, wherein the one or more static properties comprise one or more hardware capability properties of the available service option.

8. The computer-implemented method of claim 5, wherein:
the one or more current operational trends are determined based at least in part on one or more second historic operational trends with respect to one or more dynamic properties of the available service option during the current time interval,
the one or more second historic operational trends are determined based at least in part on historic operational data, and
the one or more dynamic properties are determined based at least in part on the real-time operational data.

9. The computer-implemented method of claim 1, wherein the plurality of available service options associated with the patient profile are determined based at least in part on a geographic region of the medical need scenario.

10. The computer-implemented method of claim 1, wherein:
the plurality of available service options associated with the patient profile are determined based at least in part on a selection threshold condition associated with the patient profile,
the selection threshold condition is determined based at least in part on an initial severity determination associated with the medical need scenario, and
the initial severity determination is determined based at least in part on the medical need scenario.

11. The computer-implemented method of claim 1, wherein the one or more service delivery actions comprises generating a patient recommendation user interface configured to transmit information about the optimal service option to the patient computing entity.

12. The computer-implemented method of claim 1, wherein the one or more service delivery actions comprises generating a patient recommendation user interface configured to transmit information about the optimal service option to the patient computing entity.

13. The computer-implemented method of claim 1, wherein the one or more service delivery actions comprises generating a provider notification user interface configured to transmit information about the patient profile to the provider computing entity.

14. The computer-implemented method of claim 1, wherein the one or more service delivery actions comprises performing an automated load balancing operation for the optimal service option by transmitting a load balancing request to the provider computing entity.

15. An apparatus for performing temporally dynamic medical event processing with respect to a medical need scenario, the apparatus comprising at least one processor and at least one memory including program code, the at least one memory and the program code configured to, with the processor, cause the apparatus to at least:
determine a medical need condition of a patient profile associated with the medical need scenario, wherein (i) the medical need condition is selected from a plurality of candidate medical need conditions, (ii) each of the plurality of candidate medical need conditions is characterized by one or more medical response expectations comprising a patient competence expectation, and (iii) determining the medical need condition is based at least in part on a patient competence state associated with the medical need scenario;
determine a temporally dynamic service quality model for each available service option of a plurality of available service options associated with the patient profile, wherein: (i) the corresponding temporally dynamic service quality model for an available service option indicates a current estimated service quality score for the available service option with respect to each candidate medical need condition of the plurality of candidate medical need conditions, and (ii) determining the temporally dynamic service quality model for an available service option comprises assigning a minimal current estimated service quality score for the available service option with respect to each candidate medical need condition of a plurality of candidate medical need conditions in response to determining that a transport capability designation of the available service option does not satisfy the patient competence expectation of the candidate medical need condition;
determine, based at least in part on the medical need condition and each temporally dynamic service quality model for an available service option of the plurality of available service options, an optimal service option of the plurality of available service options; and
perform one or more service delivery actions by communicating with at least one of a patient computing entity associated with the patient profile or a provider computing entity associated with the optimal service option.

16. The apparatus of claim 15, wherein each current estimated service quality score indicated by a temporally dynamic service quality model for an available service option of the plurality of available service options is determined based at least in part on historic operational data associated with the available service option and real-time operational data associated with the available service option.

17. The apparatus of claim 16, wherein determining the temporally dynamic service quality model for an available service option of the plurality of available service options comprises:
identifying a current time interval associated with the medical need scenario and the available service option;
determining, based at least in part on the historic operational data, a static characterization of the current time interval based at least in part on one or more historic operational trends of the available service option with respect to one or more static properties of the available service option during the current time interval;
determining, based at least in part on the real-time operational data, a dynamic characterization of the current time interval based at least in part on one or more current operational trends of the available service option;
determining, based at least in part on the static characterization of the current time interval and the dynamic characterization of the current time interval, a temporal designation of the current time interval; and
determining the current estimated service quality score for the available service option with respect to each candidate medical need condition of the plurality of candidate medical need conditions based at least in part on comparing the temporal designation of the current time interval to one or more medical response expectations of the medical need condition.

18. A computer program product for performing temporally dynamic medical event processing with respect to a medical need scenario, the computer program product comprising at least one non-transitory computer-readable storage medium having computer-readable program code portions stored therein, the computer-readable program code portions configured to:
determine a medical need condition of a patient profile associated with the medical need scenario, wherein: (i) the medical need condition is selected from the plurality of candidate medical need conditions, (ii) each candidate medical need condition of the plurality of candidate medical need conditions is characterized by one or more medical response expectations comprising a patient competence expectation, and (iii) determining the medical need condition is based at least in part on a patient competence state associated with the medical need scenario;
determine a temporally dynamic service quality model for each available service option of a plurality of available service options associated with the patient profile, wherein: (i) the temporally dynamic service quality model for an available service option of the plurality of available service options indicates a current estimated service quality score for the available service option with respect to each candidate medical need condition of the plurality of candidate medical need conditions, and (ii) determining the temporally dynamic service quality model for an available service option of the plurality of available service options comprises assigning a minimal current estimated service quality score for the available service option with respect to each candidate medical need condition of a plurality of candidate medical need conditions in response to determining that a transport capability designation of the available service option does not satisfy the patient competence expectation of the candidate medical need condition;
determine, based at least in part on the medical need condition and each temporally dynamic service quality model for an available service option of the plurality of available service options, an optimal service option of the plurality of available service options; and
perform one or more service delivery actions by communicating with at least one of a patient computing entity associated with the patient profile and a provider computing entity associated with the optimal service option.

19. The computer program product of claim 18, wherein each current estimated service quality score indicated by a temporally dynamic service quality model for an available service option of the plurality of available service options is determined based at least in part on historic operational data associated with the available service option and real-time operational data associated with the available service option.

20. The computer program product of claim 19, wherein determining the temporally dynamic service quality model for an available service option of the plurality of available service options comprises:
identifying a current time interval associated with the medical need scenario and the available service option;
determining, based at least in part on the historic operational data, a static characterization of the current time interval based at least in part on one or more historic operational trends of the available service option with respect to one or more static properties of the available service option during the current time interval;
determining, based at least in part on the real-time operational data, a dynamic characterization of the current time interval based at least in part on one or more current operational trends of the available service option;
determining, based at least in part on the static characterization of the current time interval and the dynamic characterization of the current time interval, a temporal designation of the current time interval; and
determining the current estimated service quality score for the available service option with respect to each candidate medical need condition of the plurality of candidate medical need conditions based at least in part on comparing the temporal designation of the current time interval to one or more medical response expectations of the medical need condition.
